(19) United States
(12) Patent Application Publication
(10) Pub. No.: US 2023/0313109 A1
(43) Pub. Date: Oct. 5, 2023

(54) DEVICE AND PROCESS FOR CELL CULTURE MEDIA PREPARATION AND PERFORMING CELL CULTURE

(71) Applicant: Merck Patent GmbH, Darmstadt (DE)

(72) Inventors: Jochen Bastian SIECK, Darmstadt (DE); Christian SCHULTHEISS, Darmstadt (DE)

(73) Assignee: Merck Patent GmbH, Darmstadt (DE)

(21) Appl. No.: 18/014,567

(22) PCT Filed: Jul. 5, 2021
(86) PCT No.: PCT/EP2021/068483

§ 371 Date: Jan. 5, 2023

(30) Foreign Application Priority Data
Jul. 6, 2020 (EP) 20184241.6

(51) Int. Cl. C12M 1/00; C12M 1/06; C12M 1/34

(52) U.S. Cl. CPC C12M 29/26; C12M 27/02; C12M 41/26

(57) ABSTRACT

The present invention relates to a device and processes for producing liquid media for cell cultures, whereby the liquid media are produced automatically by dissolving ingredients in water. The present invention also relates to a device for producing media used in cell cultures or a substance produced by cell cultures using a bioreactor process.

The present invention relates to devices and processes for preparing media for growth of mammalian cell cultures and for preferably continuously or semi-continuously performing such cell cultures. Especially, the present invention relates to devices and processes for producing liquid cell culture media for processes in bioreactors, which can be produced by dissolving powdered and/or granulated ingredients in water and which enable continuous or semi-continuous processing of a cell culture.

The most common cultivation modes used in biomanufacturing are batch culture, fed-batch and perfusion culture. The reason for choosing one of those technologies lies in different factors linked to the protein and/or the host. Cells are cultivated either attached on surfaces or in suspension. The easiest mode to operate is probably the batch bioreactor. After inoculation, cells grow and produce until a limitation due to media consumption is reached and cell density starts to decrease. The second very common process is fed-batch where nutrient limitations are prevented by adding highly concentrated feeds at different time points during the cultivation. The culture duration is therefore longer than in batch mode and the final productivity is increased.

A perfusion culture process permits bioreactors to run continuously over extended periods of time up to several months by constantly perfusing fresh medium through the culture, simultaneously providing fresh nutrients for the cells and removing spent media and optionally dead cells and target product while retaining high numbers of viable cells. The key advantages of perfusion technology include higher yields per bioreactor volume, increased flexibility and more consistent product quality. To achieve this, the system and the process need to be set up very carefully. Unlike batch-fed systems, perfusion systems accumulate no waste products. Expressed proteins can rapidly be removed and made available for purification—a significant advantage with proteins prone to instability. Removing spent media while keeping cells in culture can be done using different technologies like filtration, e.g. alternating tangential-flow (ATF) and standard tangential-flow filtration (TFF). Other methods include use of sedimentation devices, centrifuges or an acoustic device. Another option is to retain the cells by binding them to a surface (capillary fibers, membranes, microcarriers in fixed bed, and so on) in the bioreactor.

The benefits and the techniques of perfusion bioreactors is discussed in “Optimization of High Cell Density Perfusion Bioreactors” by D. Kompala and S. Ozturk, Cell culture technology for pharmaceutical and cell-based therapies, Taylor & Francis Group, 2006, ISBN-10: 0-8247-5334-8, pages 387-416.

A review about perfusion culture providing details about favorable set ups can be found in “Perfusion mammalian cell culture for recombinant protein manufacturing— A critical review” Jean-Marc Bielser et al., Biotechnology Advances 36 (2018) 1328-1340. A filtration-based perfusion system in which dead cells can only be removed from the system through bleeding is described in “Potential of Cell Retention Techniques for Large-Scale High-Density Perfusion Culture of Suspended Mammalian Cells”, D. Voisard, F. Meuwly, P.-A. Ruffieux, G. Baer, A. Kadouri, Cytotechnology 28: 163-175, 1998. In some perfusion processes, ultrafiltration membranes are used to retain the product in the bioreactor. Those processes are also called “concentrated fed-batch” or CFB. Concentrated fed-batch cell culture increases manufacturing capacity without additional volumetric capacity. Information about this special perfusion process can be found in William C. Yang, Daniel F. Minkler, Rashmi Kshirsagar, Thomas Ryll, Yao-Ming Huang, Journal of Biotechnology 217 (2016) 1-11.

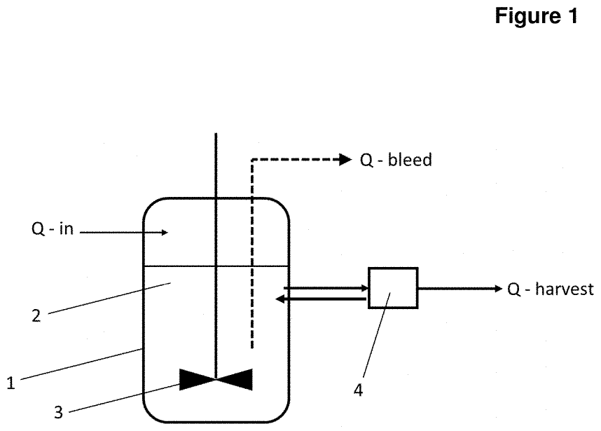

FIG. 1 shows a schematic view of a state of the art perfusion culture bioreactor. The bioreactor (1) with the cell culture (2) including the liquid cell culture medium and the cells is optionally stirred by stirrer (3). New, fresh medium can be added via Q— in, also called P. The harvest stream including cells, liquid medium and target product leaves the bioreactor (1) via the Q-harvest line. Q harvest is often called H. A cell retention device (4) retains the cells e.g. by the methods described above so that cell free or cell-reduced harvest can be collected. Typically, in perfusion culture, media is fed continuously via Q— in and harvest is removed continuously via Q— harvest. Once the cell density has reached a desired set-point excess cells need to be removed to keep a steady cell concentration and a achieve steady-state operation. This is done via the bleed stream Q-bleed, also called B. To maintain a constant volume in the bioreactor, typically Q— in=Q— harvest+Q— bleed, also called P=H+B, meaning that the volume of cell culture medium that is newly added to the bioreactor via Q-in needs to be equivalent to the volume that is removed via Q— harvest and Q— bleed. An improved perfusion cell culture bioreactor is disclosed in the non-prepublished EP 19207666.9, published as WO21089661.

Fresh medium is generated by dissolving a powder or granulate or other dry format of a mixture of nutrients and other ingredients in water. During dissolution of the ingredients pH of the watery formulation needs to be controlled and adjusted to allow the ingredients to be fully and optimally dissolved without influencing the other ingredients. This is controlled by laboratory technicians or other qualified staff. Dissolution is controlled by observing turbidity of the watery formulation to get an impression on how much of the ingredients is dissolved. The manufacturer of such powders and granulates define, how they are supposed to be dissolved correctly. For example, such a recipe is given in the product information of EX-CELL® Advanced HD Perfusion Medium (Lit. No. DS3980EN00; 2017-04512; published 04/2017) by Merck KGaA in Europe and Asia and MilliporeSigma in the U.S. and Canada.

The present state of the art for technical liquefaction of cell culture media in powdered form is purely manually performed by staff following technical mixing instructions. These mixing instructions usually contain stirring processes, changes of pH, addition of liquid and solid ingredients and holding times. Preparation of cell culture media differs from common dissolution of single component powders in water in that cell culture media are multicomponent mixtures, which contain ingredients that have strongly differing chemical and physical features, making the dissolution process complex.

Media preparation is a “core operation” within biomanufacturing facilities. For multi-product, rapid-turnover facilities, media preparation and storage can become a bottleneck. Complexity lies in scheduling and delivering media to the process on time and at the required specifications.

The state of the art has the disadvantages, that the staff always has to be available to be able to control the preparation of fresh medium. In addition, there is a risk of preparing varying batches of fresh medium, because the turbidity of the watery formulation and the times at which ingredients are added are not standardized and thus might differ from batch to batch. This might result in changes of the growth rate of cell cultures in the bioreactor using the fresh medium. Furthermore, the production of the cell cultures in the bioreactors might be interrupted due to the time needed for the preparation of new batches of fresh medium or for the time needed to wait for the required staff. A further disadvantage of the state of the art is the potential contamination of the fresh medium by human error or while opening the container for the fresh medium to control the watery formulation or to add substances to the watery formulation.

As shown the bioprocess step is typically performed manually today, which bears risks for contamination, operator error, and reproducibility. Manual media preparation for perfusion processes in industrial scale can have a significant impact on manufacturing costs today. Deviations due to operator error can severely impact the manufacturing of biopharmaceuticals, independent if basal cell culture media for perfusion, batch, fed-batch, or feed solutions are produced.

It would thus be favorable to find a way to further standardize the process of producing fresh medium and to reduce the risk of variations and contaminations in the preparation process. A further object of the present invention is to find a device which enables a more reliable preparation of the fresh medium and which allows more reproducible results. There is always a need for reducing the costs for the production, concerning money, material and labor.

Another object of the present invention is to provide the right media at the right time and at the right specification while minimizing the required labor and footprint in media preparation.

The present invention is thus directed to a system for performing cell culture comprising a bioreactor, a holding tank and a device for producing liquid media for cell cultures, whereby the liquid media are produced by dissolving ingredients in water, the device comprising a mixing vessel (10) for holding and mixing a watery formulation; an agitator (18) for mixing the watery formulation in the mixing vessel (10); at least one pH meter (24) in the mixing vessel (10) or in fluid connection with the mixing vessel (10);
optionally, but preferably at least one dissolution sensor (26) for detecting the presence of undissolved ingredients in the watery formulation;
a dosing apparatus (30) connected to the mixing vessel (10) for filling a specific amount of at least one ingredient or of at least one mixture of ingredients into the mixing vessel (10); preferably the ingredients or mixture of ingredients are dry powder or dry granulated ingredients a water supply (40) for adding water into the mixing vessel (10);
a base supply (46) for adding a specific amount of a base or a watery base to the mixing vessel (10);
an acid supply (42) for adding a specific amount of an acid or a watery acid to the mixing vessel (10);
a flow generating apparatus (48) for generating or allowing a flow of the watery formulation from the mixing vessel; preferably the flow is a flow from the mixing vessel to a holding tank or to a bioreactor;
a control system (50) connected to the pH meter (24) and, if present, to the dissolution sensor (26), such that the measured values of the pH meter (24) and the dissolution sensor (26) are accessible by the control system (50), the control system (50) being connected to the water supply (40) to control the amount of water being filled into the mixing vessel (10), the control system (50) being connected to the base supply (46) to control the amount of base or watery base being filled into the mixing vessel, the control system (50) being connected to the acid supply (42) to control the amount of acid or watery acid being filled into the mixing vessel (10), the control system (50) being connected to the dosing apparatus (30) to control the amount of the at least one ingredient or the at least one mixture of ingredients being filled into the mixing vessel (10), whereby the control system (50) is programmed to control the dosing apparatus (30), the water supply (40), the base supply (46), the acid supply (42), and preferably the flow generating apparatus (48), depending from the measured values of at least one of the pH meter (24) and, if present, the dissolution sensor (26).

The dosing apparatus is used to precisely dose at least one ingredient or at one mixture of ingredients into the mixing vessel. It comprises at least a container and an outlet. Preferably the container is a single use bag comprising the preferably solid at least one ingredient or mixture of ingredients. The dosing apparatus preferably further comprises a weight sensor to measure the weight of the container so that an exact amount of ingredients can be measured and filled from the dosing apparatus into the mixing vessel. The dosing apparatus is preferably located above the mixing vessel. Its outlet is connected to the mixing vessel directly or via a tube for transporting solid ingredients to the mixing vessel. The dosing apparatus preferably comprises a valve to enable and stop as well as adjust the flow rate of the at least one solid ingredient or mixture of ingredients to the mixing vessel. This valve and the weight sensor are preferably linked to the control system so that the control system can control the valve based on the information received from the weight sensor.

The device can also comprise a liquid supply in addition to the water, base, acid and buffer supply for the supply of other liquids like liquid cell culture supplements which cannot be added as part of the solid ingredients. The ingredients usually are nutrients, which are required to or which help to grow the cell cultures, in particular mammalian cell cultures. They are typically solid ingredients, e.g. in form of powders, compactates, pellets, tablets, granular material, e.g. wet granulated material, and/or condensed powder particles, whereby powder, compactate, pellets and/or granular material are preferred.

The at least one pH meter can be at least one common pH probe.

The produced liquid medium is a watery formulation containing the at least one ingredient or the at least one mixture of ingredients dissolved in water. Preferably the device comprises at least one sterile filter, through which the flow from the mixing vessel is conductible, whereby preferably the device comprises a multitude of sterile filters, which are interchangeable, particular preferable automatically interchangeable depending from the amount of flow through one of the sterile filters, which is actually used, or depending from the flow resistance of the flow of the solution from the mixing vessel required to uphold a flow through the actually used sterile filter.

The device may include one or more sensors or probes for detecting one or more operational parameters in real-time including, but not limited to, a state of inlet ports into the mixing vessel, a state of outlet ports out of the mixing vessel, a volumetric flow sensor to measure a volumetric flow from the mixing vessel, a weight sensor to measure the weight of content in the mixing vessel and/or the amount of the at least one ingredient or of the at least one mixture of ingredients to be filled into the mixing vessel, a liquid level sensor to measure the level of liquid in the mixing vessel, at least one thermometer, an oxygen probe, a lactic acid probe, an ammonia probe, mass spectrometry, gas chromatography, combinations thereof, and the like. The weight sensor is preferably a scale.

The watery formulation is liquid. The watery formulation can be a watery solution or a watery dispersion or a mixture of watery solution and watery dispersion.

The device can comprise a flow controller for controlling a flow of the watery formulation from the mixing vessel, whereby the control system is connected to the flow controller to control the flow of solution from the mixing vessel.

The at least one dissolution sensor can preferably be a dissolution sensor for measuring the concentration of undissolved ingredients in the watery formulation.

Preferably the water supply is a water supply for adding a specific amount of water into the mixing vessel. Hereby, it is easier to adjust the water content of the watery formulation and hence the concentration of a watery solution.

It can be provided that the device comprises at least one sterile filter, through which the flow from the mixing vessel is conductible, whereby preferably the device comprises a multitude of sterile filters, which are interchangeable, particular preferable automatically interchangeable depending on the amount of flow through one of the sterile filters, which is actually used, or depending on the flow resistance of the flow of the solution from the mixing vessel required to uphold a flow through the actually used sterile filter.

By means of the at least one sterile filter it can be ensured that the watery formulation as the produced liquid media are suitable for the reproduction of cell cultures. Furthermore, it is hereby prevented that the produced medium is contaminated by bacteria which interfere with the growth of the desired cell cultures.

It can also be provided that the device comprises at least one sensor for measuring the electrical conductivity of the watery formulation, whereby the at least one sensor for measuring the electrical conductivity of the watery formulation is located in the mixing vessel and/or in a pipe for conducting the flow from the mixing vessel, whereby the control system being connected to the at least one sensor for measuring the electrical conductivity of the watery formulation, such that the measured values of the at least one sensor for measuring the electrical conductivity of the watery formulation are accessible by the control system and the control system being designed to control at least the flow generating apparatus depending on the measured values of the at least one sensor for measuring the electrical conductivity of the watery formulation.

The electrical conductivity is a measure of the concentration of ions dissolved in the watery formulation and is also a measure of the presence of the watery formulation at the position of the at least one sensor for measuring the electrical conductivity. Therefore, the at least one sensor for measuring the electrical conductivity can be used for both purposes. The pipe can be a hose having flexible walls. In fact, it is preferred for the pipe to be a flexible hose.

It can be provided that the device comprises a sensor for measuring the osmolarity of the watery formulation, whereby the sensor for measuring the osmolarity of the watery formulation is located in the mixing vessel and/or in a pipe for conducting the flow from the mixing vessel, whereby the control system being connected to the sensor for measuring the osmolarity of the watery formulation, such that the measured values of the sensor for measuring the osmolarity of the watery formulation are accessible by the control system and the control system being designed to control at least the flow generating apparatus depending from the measured values of the sensor for measuring the osmolarity of the watery formulation.

The osmolarity is a measure of the concentration of ions solved in the watery formulation and is also a measure of the watery formulation being present at the position of the at least one sensor for measuring the electrical conductivity.

It can be provided that the flow generating apparatus is or comprises a pumping device for pumping the formulation from the mixing vessel and thereby generating the flow of the formulation from the mixing vessel and/or the flow generating apparatus is or comprises a controllable valve for controlling the flow of the formulation from the mixing vessel, whereby preferably the flow of the formulation is driven by gravity and/or the pumping device.

Hereby, the flow (volume flow) of the liquid media can be controlled by regulating the pumping power or by regulating the free cross section of the controllable valve. At least the pump can be switched on and off and the controllable valve can be opened or closed.

The at least one ingredient or the at least one mixture of ingredients can be provided as a powder, compactate, pellets, tablets, granular material and/or condensed powder particles, whereby powder, compactate, pellets and/or granular material are preferred.

A compactate is a granulated form of dry powder cell culture media. Hereby, it is easily possible to dose the amount of the at least one ingredient or the at least one mixture of ingredients and thereby the concentration of these in the watery formulation in the mixing vessel.

It can be provided that the device further comprises a timing element, whereby the control system has access to the timing element and the control system is programmed to control at least one of the dosing apparatus, the agitator, the water supply, the base supply, the acid supply and the flow generating apparatus depending from a time information taken from the timing element.

By means of the timing element it is possible to ensure thorough mixing and dissolution of the at least one ingredient or the at least one mixture of ingredients in the water. For example, it can be provided that a mixing is done until pH reaches a certain value and/or the at least one dissolution sensor measures a desired level of dissolution (i.e. a desired low amount of undissolved ingredients in the watery formulation), and after that or independently a period of mixing can be performed controlled by the control system using the timing element.

It can be provided that the control system is programmed to control the agitator depending from the measured values of at least one of the pH meter and the dissolution sensor, preferably if present depending from a measured value of the at least one sensor for measuring the electrical conductivity of the watery formulation, a measured value of the sensor for measuring the osmolarity of the watery formulation and/or a time information given by the timing element.

Hereby, it can be ensured that a thorough mixing but a mixing which does not take more time than necessary can be performed to produce the liquid media in high quality.

It can be provided that the control system is programmed to produce at least two different types liquid media for the cultivation of at least two different cell cultures or different process phases of the same cell culture process.

For example, media used during cell expansion and protein production using the same cell culture might be different.

By this, more than one type of liquid media can be produced using the device according to the invention.

It can be provided that the device comprises at least one interface being connected to the control system, whereby a starting of a mixture of a new batch of liquid media is triggerable via a signal or a request to the control system via the at least one interface, whereby preferably at least one of the required volume or amount of liquid media, the composition of the liquid media and the type of liquid media is triggerable via the at least one interface to the control system.

Hereby, the preparation of liquid media can be automatically triggered by a bioreactor to ensure sufficient supply of liquid media to the bioreactor. Automatically means that the device or system performs e.g. a certain process step without direct human control, that means without a human being starting this process step. Of course at some point in time a human being has started the system or device and has thus enabled the further automatic performance of the system or device but the process steps are thereafter performed automatically triggered by the control system which initiates certain process steps, for example triggered by signals e.g. received from a signal receiving unit or by measured values from one or more sensors.

It can be provided that the device further comprises a heater and a temperature sensor, both being connected to the control system, whereby the control system is programmed to control the heater depending from a value given by the temperature sensor, whereby preferably the control system is programmed to control at least one of the dosing apparatus, the water supply, the base supply, the acid supply, the agitator and the flow generating apparatus depending from a temperature measured by the temperature sensor.

By controlling the temperature by means of the heater and the temperature sensor it is possible to further optimize the dissolution process of the at least one ingredient or of the at least one mixture of ingredients in the water or the watery formulation. Thereby, a thorough and time efficient dissolution can be obtained.

It can be provided that the device comprises at least two pH meters in the mixing vessel and/or in fluid connection with the mixing vessel.

Hereby, pH can be measured continuously and with a higher precision. One first of the pH meters can be reset by rinsing while at least one other second pH meter can be used to continue measurement of pH. In addition, a mean value of pH can be obtained by using measurement of more than one pH meter.

It can be provided that the device comprises an outlet allowing to draw a part of the flow from the mixing vessel, preferably behind the sterile filter.

By such an outlet a sample of the liquid media can be saved for quality control. The outlet can be a tapping point.

It can be provided that the inner parts of the device are functionally closed to the surrounding of the device apart from an outflow of the volume flow of the liquid media from the mixing vessel, preferably hermetically closed to the surrounding of the device apart from the outflow of the volume flow of the liquid media from the mixing vessel.

A pollution and/or contamination of the liquid media by germs and an inlet and an outlet of gases and liquids can be prevented by functionally closing and thus isolating the device from the outside.

It can be provided that the device comprises a sensor for level indication of the watery formulation inside the mixing vessel, whereby the control system being connected to the sensor for level indication, such that the measured values of the sensor for level indication are accessible by the control system and the control system being designed to control at least the flow generating apparatus depending from the measured values of the sensor for level indication, preferably the control system being designed to control at least one of the dosing apparatus, the water supply, the base supply and the acid supply depending from the measured values of the sensor for level indication.

The sensor for level indication can be used to better control the process of producing and providing the liquid media by taking information about the level of watery formulation in the mixing vessel into account, which allows conclusions about the amount of added water, base, acid and ingredients.

It can be provided that all pipes and containers coming in contact with the watery formulation of the device are single-use parts and/or covered by single-use parts, preferably all parts of the device coming in contact with the watery formulation, the water, the base or watery base, the acid or watery acid (apart from the at least one sterile filter if applicable) are single-use parts, particular preferably also all parts coming in contact with the ingredients including or not including the dosing apparatus are single-use parts.

Hereby, it can be ensured that more than one type of liquid medium can be produced using the device without having to fear that the produced liquid media are contaminated or impaired by a liquid medium produced earlier.

It can be provided that all single-use parts are made from and/or covered by plastic, whereby preferably the walls of the mixing vessel and all pipes and tubes conducting liquids are made from and/or covered by plastic.

Hereby, the single-use parts can be easily changed without high cost and the used and contaminated single-use parts can hygienically be disposed by incineration.

The single-use parts can be made from plastic material like Polyethylene, especially from HDPE (high density polyethylene) or LDPE (low density polyethylene) or LLDPE (linear low-density polyethylene). Single-use parts can be bags, pipes, hoses and/or foils. Single-use parts can be connected by gluing and/or welding. Such single-use parts are commercially available as Mobius® Bags from MilliporeSigma in the U.S. and Canada and from Merck KGaA in Europe and Asia.

It can be provided that the water supply, the base supply and the acid supply each comprise a pump and a tank, whereby each pump is separately controllable by the control system, whereby preferably the pumps are peristaltic pumps.

Hereby, the supply of water, base and acid can easily be controlled and the input of a predetermined amount of these substances can easily be controlled.

It can be provided that all valves of the device coming in contact with either of the watery formulation, the water, the base, the watery base, the acid or the watery acid are pinch valves.

Hereby, the tubes or hoses in which the watery formulation, the water, the base, the watery base, the acid or the watery acid are conducted can be completely changed also in the pinch valves. This allows a hygienical and contamination free change to new types of liquid media.

It can be provided that the mixing vessel is made of plastic, glass or stainless steel. It can be a tank, flask, vessel or also a bag. Preferably it has a volume of not less than 5 liters and not more than 1000 liters, more preferably the mixing vessel has a volume of not less than 50 liters and not more than 200 liters.

These volumes are sufficient for producing liquid media batches for bioreactors.

It can be provided that at least one of the one or more dissolution sensors for measuring the concentration of undissolved ingredients is located in a pipe connected to the mixing vessel, preferably in a loop connected to the mixing vessel.

By this arrangement, the value of the dissolution can be measured with a higher precision and a better reproducibility.

It can be provided that the agitator comprises mixing blades which are rotatable around an axis perpendicular to the mixing blades, preferably further comprising a motor driven shaft connected to the mixing blades, whereby particular preferably a motor connected to the shaft and driving the shaft connected to the mixing blades is controlled by the control system.

Hereby, the mixing process can be easily controlled and a thorough mixing of the watery formulation can be easily obtained.

In one embodiment the device is a device for continuously or semi-continuously producing liquid media for the growth of cell cultures by dissolving water-soluble ingredients in water.

It can be provided that the device comprises a volume sensor and/or a liquid level sensor to measure the volume of watery formulation in the mixing vessel, whereby the control system being connected to the volume sensor and/or the liquid level sensor to control the amount of watery formulation in the mixing vessel and being programmed to fill water and/or the at least one ingredient or the at least one mixture of ingredients into the mixing vessel depending from the measured value of the volume sensor and/or the liquid level sensor.

By use of these sensors the dissolution of the ingredients and the mixing of the watery formulation in the mixing vessel can be controlled and optimized.

It can be provided that the device comprises at least one weight sensor to measure the weight of content in the mixing vessel and/or the amount of the at least one ingredient or of the at least one mixture of ingredients to be filled into the mixing vessel by the dosing apparatus, whereby the control system being connected to the at least one weight sensor to control the amount of watery formulation in the mixing vessel and being programmed to fill water and/or the at least one ingredient or the at least one mixture of ingredients into the mixing vessel depending from the measured value of the at least one weight sensor, and/or the control system being connected to the at least one weight sensor to control the weight of the at least one ingredient or of the at least one mixture of ingredients to be filled into the mixing vessel by the dosing apparatus and being programmed to fill water and/or additional of the at least one ingredient or the at least one mixture of ingredients into the mixing vessel depending from the weight measured by the at least one weight sensor.

At least one of the at least one weight sensor can be part of the dosing apparatus, to measure the weight of any one of the at least one ingredient or of the at least one mixture of ingredients to be filled into the mixing vessel by the dosing apparatus.

In a preferred embodiment the device comprises two weight sensors. One weight sensor measures the weight content of the mixing vessel and the other weight sensor measures the weight of the at least one ingredient to be added to the mixing vessel via the dosing apparatus. Those two weight sensors can have different working ranges as the weight of the content of the mixing vessel is typically in the range of 200 to 2000 kg and the weight of the at least one ingredient is typically between 5 to 100 kg.

Hereby, the concentration of the at least one ingredient or of the at least one mixture of ingredients in the watery formulation can be adjusted precisely.

It can be provided that the at least one dissolution sensor for measuring the concentration of undissolved ingredients is at least one turbidity sensor and/or at least one opacimeter and/or at least one scattered light sensor or a similar sensor.

These sensors are commercially available and are suitable for measuring the amount of undissolved ingredients in the watery formulation. For example, at least one AS56-N Turbidity Probe from optek-Danulate GmbH (Essen, Germany) can be used as the at least one turbidity sensor. Inline turbidity probes are also available from Pyxis (models ST-730, ST-730B, ST-730SS, ST-731, and SZ-735) which may be used as the at least one turbidity sensor.

In one embodiment the device further comprises a buffer solution supply for adding a specific amount of one or more buffer solutions to the mixing vessel, whereby the control system being connected to the buffer solution supply to control the amount of the one or more buffer solution being filled into the mixing vessel, whereby preferably the control system is programmed to control the buffer solution supply depending from the measured values of at least one of the pH meter and the dissolution sensor.

Hereby a buffering of the watery formulation can be achieved. For example, a Ringer-solution can be used as the buffer solution. A combination of bicarb and Good's buffers (like HEPES, MOPS) can be used as buffer solutions. But suitable buffer solutions can also contain all components of Ringer solutions.

A buffer tank can be arranged in a fluid line between the mixing vessel and a bioreactor which is fed by the device.

It can be provided that the device allows an automated filtration functionality of the watery formulation.

pH meters are commercially available. For example, an AppliSens pH+ Sensor from Applikon® BIOTECHNOLOGY can be used as pH meter or a Hanna Instruments PP pH-analysis electrode can also be used as pH meter

The device for producing liquid media for cell cultures according to the present invention can also be part of a system for performing a cell culture which is used for culturing cells and optionally for therewith producing a substance, e.g. a biopharmaceutical. The present invention is thus further directed to a system for performing cell cultures and/or producing a substance produced by a cell culture comprising the device for producing liquid media for cell cultures and a bioreactor, especially a perfusion reactor, whereby the flow of the watery formulation from the mixing vessel generated or allowed by the flow generating apparatus flows into the bioreactor, directly or indirectly via a holding tank, whereby the control system is connected to a signal receiving unit, which is able to receive a signal from the bioreactor, and the control system is programmed to control the flow of the watery formulation in response to the signal received from the bioreactor via the signal receiving unit, whereby preferably at least one sterile filter is arranged in a pipeline for conducting the flow of liquid media from the mixing vessel to the bioreactor. The device for producing liquid media for cell cultures is thus connected with the bioreactor via at least a pipeline or tube between the mixing vessel and the bioreactor which comprises at least one sterile filter and which might be interrupted by a holding tank.

Thereby, the device is able to make use of the liquid media to directly perform a cell culture and/or produce a substance produced by these cell cultures on demand. The bioreactor can comprise a signal sending unit which can send a signal to the signal receiving unit. Preferably a controller of the bioreactor is programmed to send a signal via the signal sending unit if the amount of watery formulation drops below a certain value or generally if fresh watery formulation is needed to uphold the production of cell cultures in the bioreactor. Several different criteria can be used to define if fresh watery formulation is needed in the bioreactor, such as the remaining volume of fresh watery formulation in the bioreactor, the speed of consumption of watery formulation, the progress of the cell culture production and combinations thereof.

Especially if cell culture processes are used in biopharma production the processes have to run reliably and favorably. In addition, some products require continuous or semi-continuous operation due to product stability of for process economic reasons. The system and the process of the present invention provide for automated media reconstitution and provision of fresh medium in the amount, composition and quality that is needed for the respective cell culture. By linking the control system to the bioreactor, the holding tank and the device for producing liquid media for cell cultures the control system can receive signals indicating if the amount of watery formulation in the bioreactor and/or the holding tank drops below a certain value or generally if fresh watery formulation is needed. The control system is programmed such that the respective signals from the bioreactor and/or the holding tank automatically trigger, with a specific timing of preparation and/or adjusted volume of preparation of the watery formulation in the mixing vessel and/or flow into the holding tank and/or the production of new watery formulation in the device for producing liquid media for cell cultures so that at any time during the cell culture process sufficient watery formulation is available. The system runs automatically and besides programming of the control system and providing sufficient reagents for the device for producing liquid media for cell cultures, the system once started can run without human interaction for more than 12 hours, preferably for more than 24 hours.

Preferably the device for producing liquid media for cell cultures, the holding tank and the bioreactor are connected via a tube or pipeline such that a flow of watery formulation can be initiated from the mixing vessel to the holding tank and from the holding tank to the bioreactor. Typically, the control system can initiate the action of the respective pumps and/or valves to enable the flow and stop the flow. Preferably, the control system is linked to signal sending units in the bioreactor and the holding tank. Such signal sending units may be sensors for level indication of the filling height of the bioreactor and/or the holding tank. The signal sending units may also be coupled to such sensors. They may also be other types of sensors or linked to other types of sensors suitable to measure or detect the demand for fresh medium like volume sensors, conductivity sensors, turbidity sensors, flow sensors, sensors for the concentration of certain ingredients of the watery formulation, pH sensors, weight sensors, pressure sensors, etc. The signal sending units and the sensors in the bioreactor and in the holding tank may be identical or different. Typically the system is programmed such that if the sensors measure a value that is below or above a certain threshold, the signal sending units send a signal to the control system, more precisely to a signal receiving unit of the control system, and the control system automatically initiates an action. Such an action may be the flow of watery formulation from the holding tank to the bioreactor. It may also be the flow of watery formulation from the mixing vessel to the holding tank. It may also be the start of production of new watery formulation in the device for producing liquid media for cell cultures. Preferably the bioreactor is a perfusion bioreactor. Preferably the holding tank has a volume between 500 and 2000 L. Preferably the liquid flow from the mixing vessel to the holding tank and from the holding tank to the bioreactor is controlled by peristaltic pumps and/or pinch valves.

The system of this invention is programmed such that parts of it like for example the device for producing liquid media could also be temporarily in an idle state, until the bioreactor, the holding tank and/or the control system signals medium demand to the system. The device would then be triggered by the system, more precisely by the control system, to go from the idle state to a productive state and prepare the required amount of cell culture medium on demand. Human interaction is typically not required. The system is programmed to automatically perform all steps required to produce and provide the medium to the bioreactor.

The problems solved by the present invention are also solved by a process for performing cell culture.

The process is preferably operated in a continuous or semi-continuous mode. The process comprises culturing cells in a bioreactor. For example in perfusion cell culture fresh cell culture medium is continuously or semi-continuously added to the bioreactor. This requires also continuous availability of fresh culture medium. According to the present invention this is done by continuously or semi-continuously producing fresh medium in the device for producing liquid media for cell culture which is part of the system for performing cell culture. Typically, the production is done semi-continuously so that at any time during the cell culture process fresh medium is available in the holding tank and the fresh medium can then be continuously or semi-continuously provided to the bioreactor.

Fresh medium is liquid cell culture medium that is sterile filtered and ready for use in cell culture.

This process comprises a process for producing liquid media for cell culture, using a device comprising a mixing vessel for holding and mixing a watery formulation, at least one pH meter in the mixing vessel or in fluid connection with the mixing vessel, and optionally at least one dissolution sensor for detecting the presence of undissolved ingredients in the watery formulation, a dosing apparatus connected to the mixing vessel for filling a specific amount of at least one ingredient or of at least one mixture of ingredients into the mixing vessel, and a control system connected to the pH meter and the dissolution sensor.

The process for semi-continuously producing liquid media for cell culture comprises the method steps of
A) Filling water and a specific amount of at least one of the at least one ingredient or at least one of the at least one mixture of ingredients into the mixing vessel and mixing them therein to a watery formulation;
B) Once or repeatedly measuring pH by the at least one pH meter and detecting the presence of undissolved ingredients by the at least one dissolution sensor of the watery formulation in the mixing vessel by means of the control system;
C) Automatically filling at least once a specific amount of water, base, watery base, acid, watery acid or one or more buffer solutions into the mixing vessel depending on the measured pH value and/or the presence of undissolved ingredients by means of the control system;
D) After all of the required at least one ingredient or the at least one mixture of ingredients have been filled into the mixing vessel and mixed to a final watery formulation, providing a volume flow of the final watery formulation from mixing vessel controlled by the control system after the control system determines that the at least one ingredient or the at least one mixture of ingredients have been dissolved in the final watery formulation depending from the measured concentration of undissolved ingredients.

It can be provided that the process is performed using a device according to the present invention. The process shares the advantages of the devices.

It can be provided that the process further comprises the method step C2) Automatically filling a specific amount of an additional of the at least one of the at least one ingredient or of an additional of at least one of the at least one mixture of ingredients into the mixing vessel controlled by the control system depending on the measured pH value and/or the measured concentration of undissolved ingredients before method step D).

Hereby, different ingredients can be added to the watery formulation at different times and into watery formulations having different physical properties such as pH value, temperature or the like. This can help to solve ingredients without negatively affecting them.

It can be provided that the water, the base, the watery base, one or more buffer solutions, the acid or the watery acid are filled into the mixing vessel controlled by the control system by means of pumps and/or valves.

Thereby, an automatically dissolution process can be controlled by the control system.

It can be provided that the device comprises at least one sensor for measuring the electrical conductivity of the watery formulation, whereby in step B) the control system controls the electrical conductivity of the watery formulation and in step C) the specific amount of water, base, watery base, acid or watery acid is filled into the mixing vessel depending on the measured value of the electrical conductivity and/or in step D) the control system determines if the at least one ingredient or the at least one mixture of ingredients have been dissolved in the watery formulation depending from the measured electrical conductivity.

Hereby, it can be ensured that a thorough mixing but a mixing which does not take more time than necessary can be performed to produce the liquid media in high quality.

Provision may be made that in step C2) the point of time in which the specific amount of the additional of the at least one of the at least one ingredient or of the additional of at least one of the at least one mixture of ingredients is automatically filled into the mixing vessel is determined on the measured value of the electrical conductivity and controlled by the control system.

It can be provided that the device comprising a sensor for measuring the osmolarity of the watery formulation, whereby
in step B) the control system controls the osmolarity of the watery formulation and in step C) the specific amount of water, base, watery base, acid or watery acid is filled into the mixing vessel depending on the measured value of the osmolarity and/or
in step D) the control system determines if the at least one ingredient or the at least one mixture of ingredients have been dissolved in the watery formulation depending from the measured osmolarity.

Hereby, it can be ensured that a thorough mixing but a mixing which does not take more time than necessary can be performed to produce the liquid media in high quality.

Provision may be made that in step C2) the point of time in which the specific amount of the additional of the at least one of the at least one ingredient or of the additional of at least one of the at least one mixture of ingredients is automatically filled into the mixing vessel is determined on the measured value of the osmolarity and controlled by the control system.

It can be provided that the device comprises a volume sensor and/or a liquid level sensor to measure the volume of liquid in the mixing vessel, whereby
in step B) the control system controls the volume and/or the liquid level of the watery formulation and in step A) the specific amount of water and the at least one of the at least one ingredient or the at least one of the at least one mixture of ingredients filled into the mixing vessel is controlled depending on the measured value of the volume and/or the liquid level and/or
in step B) the control system controls the volume and/or the liquid level of the watery formulation and in step C) the specific amount of water, base, watery base, acid or watery acid is filled into the mixing vessel depending on the measured value of the volume and/or the liquid level.

By use of these sensors the dissolution and the mixing of the watery formulation in the mixing vessel can be controlled and optimized.

It can be provided that the device comprises a weight sensor to measure the weight of content in the mixing vessel, whereby
in step B) the control system controls the weight of the watery formulation of the content in the mixing vessel and/or
in step C) the specific amount of water, base, watery base, acid or watery acid filled into the mixing vessel is controlled depending on the measured value of the measured weight of the content in the mixing vessel.

Hereby, the concentration of the at least one ingredient or of the at least one mixture of ingredients in the watery formulation can be adjusted precisely.

It can be provided that the device comprises a weight sensor to measure the amount of the at least one ingredient or of the at least one mixture of ingredients to be filled into the mixing vessel by the dosing apparatus, whereby
in step A) the specific amount of water and the at least one of the at least one ingredient or the at least one of the at least one mixture of ingredients filled into the mixing vessel is controlled depending on the measured value of the measured amount of the at least one ingredient or of the at least one mixture of ingredients to be filled into the mixing vessel by the dosing apparatus.

Hereby, the concentration of the at least one ingredient or of the at least one mixture of ingredients in the watery formulation can be adjusted precisely.

It can be provided that the volume flow from mixing vessel is conducted through at least one sterile filter, whereby preferably the volume flow passing through the at least one sterile filter is controlled by the control system and the control systems automatically changes or cleans the at least one sterile filter or gives a signal to change or clean the at least one sterile filter if the measured volume flow drops below a predefined value.

By means of the at least one sterile filter it can be ensured that the watery formulation as the produced liquid media are suitable for the reproduction of mammalian cell cultures. Furthermore, it is hereby prevented that the produced medium is contaminated by bacteria which interfere with the growth of the desired cell cultures.

It can be provided that the process is started by the control system after receiving a signal or a request for fresh medium received via an interface, whereby preferably the signal or the request is sent by a bioreactor receiving the volume flow of watery formulation from mixing vessel.

Hereby, the preparation of liquid media can be automatically triggered on demand by a bioreactor to ensure sufficient supply of liquid media to the bioreactor.

It can be provided that single-use parts like pipes, bags, casings and claddings, which come into contact with the watery formulation, are removed and exchanged for new single-use parts before a new process for producing a new type of liquid medium is started.

Hereby, it can be ensured that more than one type of liquid media can be produced using the device without having to fear that the produced liquid media are contaminated or impaired by an earlier liquid medium.

The process for performing cell culture preferably comprises the following method steps:
Providing a system for performing cell culture comprising a bioreactor, a holding tank and a device for producing liquid media for cell cultures
Performing cell culture in a bioreactor
Continuously or one or several times during the cell culture performing the process for producing liquid media for cell culture, as described above, using a device for automatically producing sterile filtered liquid media for cell culture according to the present invention
Continuously or one or several times during the cell culture flowing watery formulation, which typically is liquid medium for cell culture produced in the device for producing liquid media for cell culture from the mixing vessel of the device to the holding tank and/or from the holding tank to the bioreactor.

In a preferred embodiment the process involves the control system of the system for performing cell culture receiving signals from a signal sending unit in the bioreactor and/or in the holding tank whereby such signals trigger the control unit to initiate performing the process for producing liquid media for cell culture and/or the flow of watery formulation from the mixing vessel to the holding tank and/or from the holding tank to the bioreactor.

It can be provided that the process is a process for perfusion of cell culture, further comprising culturing cells in a bioreactor with fresh liquid media inlet and a harvest outlet, comprising the method steps of
i. continuously or one or several times during the cell culture process fresh liquid media from the device is inserted into the bioreactor via the perfusion inlet; and
ii. continuously or one or several times during the cell culture process harvest is removed from the bioreactor via the harvest outlet.

The process for perfusion of cell culture can be steadily and constantly kept running by making use of the process for producing liquid media for perfusion of cell culture according to the present invention.

It can be provided that the process steps i and ii are regulated such that the volume of the cell culture in the bioreactor is kept at a constant level.

Hereby, the process runs stable.

It can be provided that a sensor for measuring the liquid level in the bioreactor automatically sends a signal or a request for fresh liquid media to the control system if the level of liquid media drops below a predefined value or if harvest is removed from the bioreactor, whereby the control system starts preparation of fresh liquid media upon receiving the signal or the request.

Hereby, it can be ensured, that the enough liquid medium is always present in the bioreactor to produce cell cultures.

The present invention is based on the surprising findings that the process of producing fresh medium can be automated by means of a control system having access to at least one pH meter measuring the pH value and to at least one dissolution sensor for detecting the presence of undissolved ingredients in the watery formulation, while the control system is programmed to add substances and/or ingredients to the watery formulation during the preparation process based on the measured values and is further programmed to provide a flow of the watery formulation, which can be used to feed a bioreactor. Furthermore, the device and the process according to the present inventions allows to keep up continuous production of cell culture by allowing in time preparation of fresh liquid medium in form of watery formulation or watery solution. The device and the process allow the preparation of the watery formulation in high and reproducible quality and high purity.

The invention allows automated preparation of sterile filtered liquid media for the cultivation of mammalian cells (cell culture media) from a dry powder medium or compactates thereof or from granulates and water. The risks for contamination, operator error, and reproducibility of the processes according to the state of the art are overcome by a device according to the present invention in form of a machine that automatedly doses water, other liquids, powder, granulate, compactates or other dry formats of ingredients, and/or buffer solution and adjusts the pH of the watery formulation following media formulation dependent recipes of pH setpoints and mixing periods. Furthermore, the device and the process according to the present invention allow to perform online quality control before a sterile filtration. The process can be followed by automated cleaning of the device and/or the filters. The device and the process are designed to allow supplying a continuous perfusion bioreactor with freshly prepared medium on demand in a fully automated manner without operator interaction for several days.

The present invention allows more cost-efficient, reproducible and thus safer biopharmaceutical drug manufacturing. The invention is directed to allow to perform the dissolution process automatically to simplify the complex dissolution process for the ingredients of multicomponent cell culture media. This is achieved by replacing each and every single manual step of the process by an automated process performed by technical devices and means and by performing these steps by these technical devices and means. The benefit of the present invention is to accelerate the whole process of preparing cell culture media by using and evaluating specific sensors. The automation allows the liquification of cell culture media on demand.

The device and the process according to the present invention allow to reduce operator work for perfusion media preparation, allows to increase reproducibility and reduce out of stock events and human error through automation and ensures that media are always prepared according to specification.

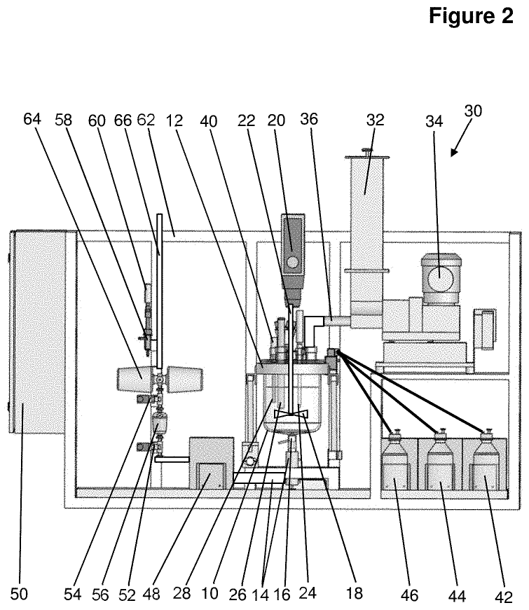

Further embodiments of the invention will now be explained with reference to three schematic FIGS. 2 to 4 below, however without limiting the invention. Wherein:

FIG. 2 shows a schematic view on a device for producing liquid media for cell cultures according to the invention;

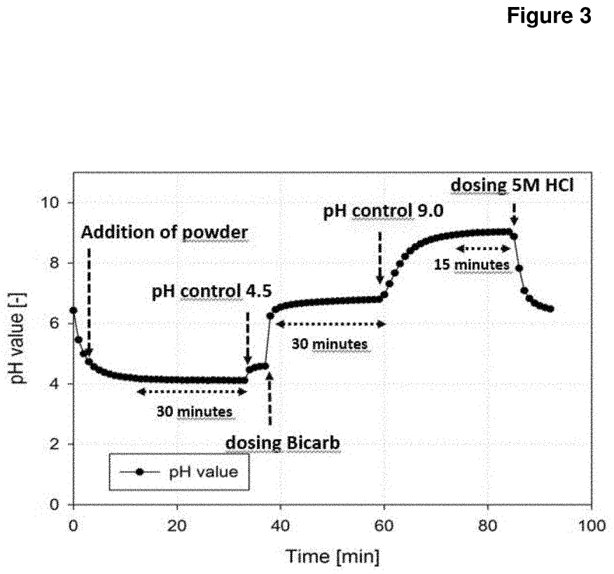

FIG. 3 shows a measured pH of a watery formulation during a process for producing liquid media for cell culture;

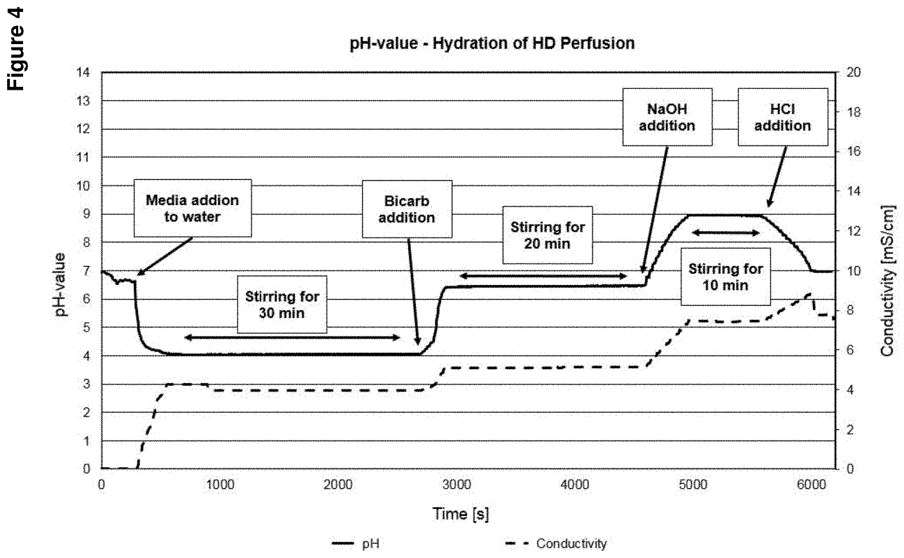

FIG. 4 shows a measured pH and a measured electrical conductivity of a watery formulation during a process for producing liquid media for cell culture; and

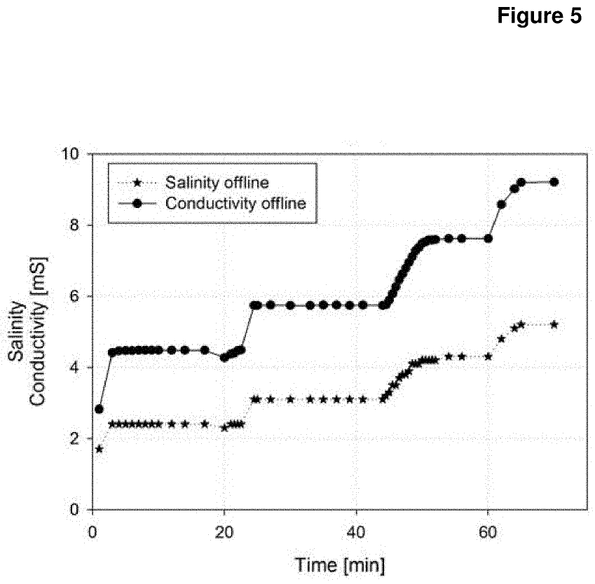

FIG. 5 shows measured salinity and electrical conductivity of a watery formulation during a process for producing liquid media for cell culture.

A cell culture is any setup in which cells are cultured. A cell culture can typically be performed in a bioreactor.

A bioreactor can be any container suitable for the culture of cells, such as a bottle, tube, vessel, bag, flask and/or tank. Typically, the container can be sterilized prior to use. A cell culture can typically be performed by incubation of the cells in an aqueous cell culture medium under suitable conditions for growth and/or maintenance of the cells such as suitable temperature, pH, osmolality, aeration, agitation, etc. which limit contamination with microorganisms from the environment. A person skilled in the art is aware of suitable incubation conditions for culturing of cells. A bioreactor used according to the present invention is preferably a bioreactor suitable for perfusion cell culture.

The aqueous cell culture medium is liquid media in form of the watery formulation or watery solution. The watery formulation and watery solution can be produced by the device for producing liquid media and the process according to the invention as the final product coming from the device and provided in the flow controlled, generated or allowed by a flow generating apparatus, preferably after filtering the watery solution with a sterile filter.

A bioreactor system suitable to be used according to the present invention comprises the bioreactor and additional equipment that is necessary to run a cell culture in said bioreactor like one or more of the following
devices for stirring
devices for supply and discharge of components to and from the bioreactor, e.g. tubes, pumps, valves, storage tanks
a cell retention device (see above)
a system for monitoring bioreactor volume, e.g. a bioreactor balance, level sensors etc.
devices for controlling and maintaining temperature, osmolarity, aeration, agitation, etc.
a computer control system for automated or partially automated operation of the cell culture bioreactor

The terms liquid medium and cell culture medium are synonymously used and further the term culture medium is also synonymously used in the present invention. A liquid medium or cell culture medium according to the present invention can be any mixture of components which maintains and/or supports the in vitro growth of cells and/or supports or maintains a particular physiological state. The same can be true for the watery formulation according to the present invention, which is produced by the device and by the process. The watery solution produced by the device for producing liquid media and the process according to the present invention can be a cell culture medium.

The liquid medium or cell culture medium might comprise undefined components, such as plasma, serum, embryo extracts, or other non-defined biological extracts or peptones. The liquid medium or cell culture medium might also, preferably, be a chemically defined medium. The liquid medium or cell culture medium can comprise all components necessary to maintain and/or support the in vitro growth of cells or be used for the addition of selected components in combination with or not in combination with further components that are added separately (media supplement). The components of a liquid medium or a cell culture medium are also called cell culture media ingredients or ingredients for the watery formulation.

The cell culture devices and processes according to the present invention can be designed to be suitable to grow or maintain/support the growth of prokaryotic cells like bacterial cells as well as eukaryotic cells like yeast, fungi, algae, plant, insect and/or mammalian cells and, optionally, archaea. Preferred cells are mammalian cells.

Chemically defined cell culture media or liquid media and chemically defined watery formulation can be cell culture media and watery formulation comprising of chemically well characterized ‘defined’ raw materials. This means that the chemical composition of all the chemicals used in the media is known. The chemically defined media and watery formulation do not comprise of chemically ill-defined substances like chemically ill-defined yeast, animal or plant tissues; they do not comprise peptones, feeder cells, serum, ill-defined extracts or digests or other components which may contribute chemically poorly defined proteins and/or peptides and/or hydrolysates to the media. In some cases, the chemically defined medium and formulation may comprise proteins or peptides which are chemically defined—one example is insulin.

A liquid (cell culture) medium and a watery formulation are typically produced by dissolving powdered and/or granulated ingredients or mixtures of ingredients in water.

A powdered or powdery ingredient or a dry powder ingredient or a dehydrated culture medium is typically resulting from a milling process or a lyophilization process. That means the powdered ingredient can typically be a finely granular, particulate medium—not a liquid medium. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated. A granulated ingredient, e.g. dry granulated can be obtained by roller compaction or wet granulated by fluidized bed spray granulation. Such an ingredient can also be prepared by spray drying or lyophilization. The pH of the watery formulation or liquid (cell culture) medium prior to addition of cells is typically between pH 2 and 12, more preferable between pH 4 and 10, even more preferable between pH 6 and 8 and most preferable between pH 6.5 to 7.5 and ideally between pH 6.8 to 7.3.

The ingredients used for producing the watery formulation and thus the liquid medium typically comprise at least one or more saccharide components, one or more amino acids, one or more vitamins or vitamin precursors, one or more salts, one or more buffer components, one or more co-factors and one or more nucleic acid components (nitrogenous bases) or their precursors and derivatives. The ingredients for the watery formulation and thus the liquid medium may also comprise chemically defined biochemicals such as recombinant proteins, e.g. rInsulin, rBSA, rTransferrin, rCytokines, etc.

The ingredients and hence the watery formulation and the liquid medium may also comprise sodium pyruvate, highly purified and hence chemically well-defined extracts, fatty acids and/or fatty acid derivatives and/or poloxamer product components (block copolymers based on ethylene oxide and propylene oxide) in particular Poloxamer 188 sometimes called Pluronic F 68 or Kolliphor P 188 or Lutrol F 68 and/or surface active components such as chemically prepared non-ionic surfactants. One example of a suitable non-ionic surfactants is difunctional block copolymer surfactants terminating in primary hydroxyl groups also called poloxamers, e.g. available under the trade name Pluronic® from BASF, Germany. Such poloxamer product components are in the following just called poloxamer or pluronic. Chelators, hormones and/or growth factors may also be added.

Other ingredients the watery formulation and the liquid medium may comprise of are the pure compounds, salts, conjugates, and/or derivatives of lactic acid, thioglycolic acid, thiosulphates, tetrathionate, diaminobutane, myo-inositol, phosphatidylcholine (lecithin), sphingomyelin, iron containing compounds (including compounds with iron-sulphur-clusters), uric acid, carbamoyl phosphate, succinic acid, thioredoxin(s), orotic acid, phosphatidic acid, polyamines (such as putrescine, spermidine, spermine and/or cadaverine), triglycerides, steroids (including but not limited to cholesterol), metallothionine, oxygen, glycerol, urea, alpha-ketoglutarate, ammonia, glycerophosphates, starch, glycogen, glyoxylate, isoprenoids, methanol, ethanol, propanol, butanol, acetone, lipids (including but not limited to those in micelles), tributyrin, butyrin, cholic acid, desoxycholic acid, polyphosphate, acetate, tartrate, malate and/or oxalate.

Saccharide ingredients are all mono- or di-saccharides, like glucose, galactose, ribose or fructose (examples of monosaccharides) or sucrose, lactose or maltose (examples of disaccharides) or derivatives thereof like sugar alcohols. Saccharide components may also be oligo- or polysaccharides.

Examples of amino acids according to the invention are particularly the proteinogenic amino acids, especially the essential amino acids, leucine, isoleucine, lysine, methionine, phenylalanine, threonine, tryptophan and valine, as well as the non-proteinogenic amino acids such as D-amino acids. Amino acid precursors and analogues can also be included, like S-sulfocysteine and phophotyrosine as well as the respective keto acids or lactoyl aminoacids.

Examples of vitamins are Vitamin A (Retinol, retinal, various retinoids, and four carotenoids), Vitamin B1 (Thiamine), Vitamin B2 (Riboflavin), Vitamin B3 (Niacin, niacinamide), Vitamin B5 (Pantothenic acid), Vitamin B6 (Pyridoxine, pyridoxamine, pyridoxal), Vitamin B7 (Biotin), Vitamin B9 (Folic acid, folinic acid), Vitamin B12 (Cyanocobalamin, hydroxycobalamin, methylcobalamin), Vitamin C (Ascorbic acid) (including phosphates of ascorbic acid), Vitamin D (Ergocalciferol, cholecalciferol), Vitamin E (Tocopherols, tocotrienols) and Vitamin K (phylloquinone, menaquinones). Vitamin precursors and analogues can also be included.

Examples of salts are components comprising inorganic ions such as bicarbonate, calcium, chloride, magnesium, phosphate, potassium and sodium or trace elements such as Co, Cu, F, Fe, Mn, Mo, Ni, Se, Si, Ni, Bi, V and Zn. Examples are copper(II) sulphate pentahydrate (CuSO4.5 H2O), sodium chloride (NaCl), calcium chloride (CaCl2.2 H2O), potassium chloride (KCl), iron(II)sulphate, sodium phosphate monobasic anhydrous (NaH2PO4), magnesium sulphate anhydrous (MgSO4), sodium phosphate dibasic anhydrous (Na2HPO4), magnesium chloride hexahydrate (MgCl2.6 H2O), zinc sulphate heptahydrate (ZnSO4.7 H2O).

Examples of buffers are carbonate, citrate, phosphate, HEPES, PIPES, ACES, BES, TES, MOPS and TRIS. A buffer solution is a watery solution of at least one buffer.

Examples of cofactors are compounds, salts, complexes and/or derivatives of thiamine, biotin, vitamin C, calciferol, choline, NAD/NADP (reduced and/or oxidized), cobalamin, vitamin B12, flavin mononucleotide and derivatives, flavin adenine dinucleotide and derivatives, glutathione (reduced and/or oxidized and/or as dimer), haeme, haemin, haemoglobin, ferritin, nucleotide phosphates and/or derivatives (e.g. adenosine phosphates), coenzyme F420, s-adenosyl methionine, coenzyme B, coenzyme M, coenzyme Q, acetyl Co-A, molybdopterin, pyrroloquinoline quinone, tetrahydrobiopterin.

Nucleic acid components are the nucleobases, like cytosine, guanine, adenine, thymine, uracil, xanthine and/or hypoxanthine, the nucleosides like cytidine, uridine, adenosine, xanthosine, inosine, guanosine and thymidine, and the nucleotides such as adenosine monophosphate or adenosine diphosphate or adenosine triphosphate, including but not limited to the deoxy- and/or phosphate derivatives and/or dimers, trimers and/or polymers thereof, like RNA and/or DNA.

Specific ingredients may be added which improve the physico-chemical properties of the watery formulation and the liquid media, like but not limited to, increasing clarity and/or solubility of the watery formulation and/or one or more of its components, without significantly negatively affecting the cell growth properties at the concentrations used. Such components include but are not limited to chelating agents (e.g. EDTA), antioxidants, detergents, surfactants, emulsifiers (like polysorbate 80), neutralizing agents, (like polysorbate 80), micelle forming agents, micelle inhibiting agents and/or polypropylene glycol, polyethylene alcohol and/or carboxymethylcellulose.

The terms “perfusion” or “perfusion process” refers to a cell culture process used to produce a target product, e.g., an antibody or recombinant protein, in which a high concentration of cells within a bioreactor receive fresh growth medium continuously or one or more times during cell culture whereby the spent medium which may contain a target product is harvested, which means removed from the bioreactor continuously or one or more times during cell culture. Preferably, fresh liquid medium or watery formulation is continuously fed into the bioreactor and spent medium which may contain the target product is harvested continuously.

An exemplary bioreactor suitable for perfusion cell culture comprises a cell retention device to keep the cells in the bioreactor during harvesting. This cell retention device can be acoustic, alternating tangential flow (ATF), a settler, a centrifuge, and the like. In some examples, disposable, reusable or semi-disposable bioreactors may be used. Any combination of hardware design may be used. In one example, a disposable cell retention device may be used. In some embodiments, disposable conduits, tubing, pumps, bag assemblies and cell retention devices are used instead of hard piping and reusable devices.

The mixing vessel according to the present invention and/or the bioreactor may have any suitable volume including, but not limited to, about 1 L to about 5000 L, but are not limited to this exemplary range. Certain exemplary mixing vessel volumes and/or bioreactor volumes include, but are not limited to, about 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 500, 1000, 1500, 2500, 4000 L, any intermediate volumes, and the like.

The bioreactor may include one or more inlets, also called inlet ports, for the introduction of one or more feeds (e.g., liquid medium, cell culture medium, watery formulation), chemical substances (e.g., pH buffers), anti-foam agents, and the like. It may also include one or more outlets, also called outlet ports, for the removal of cells and/or liquid from the bioreactor. Each inlet and/or outlet in the bioreactor may be provided with any suitable mechanism for initiating and conducting fluid flow through the inlet and/or outlet including, but not limited to, one or more peristaltic pumps, one or more pressurization mechanisms, and the like. Each inlet and/or outlet may be provided with any suitable mechanism for monitoring and controlling fluid flow through the inlet including, but not limited to, one or more mass flow meters, one or more flow control valves, and the like. For example, the bioreactor may include a flow control mechanism to control the flow rate of substances into and out of the bioreactor. The bioreactor may also comprise means for volume and/or level control.

The bioreactor comprises a media inlet, that may be operated at discrete times or continuously to introduce new liquid medium or watery formulation from the device for producing liquid media into the cell culture. The bioreactor can comprise one or more harvest outlets for releasing spent cell culture, cells and/or target products. A harvest outlet may comprise a flow control valve to control the rate of harvest.

The valves are positioned such that they can hinder, allow or direct the flow of the liquid medium or the watery formulation or a liquid in general. Examples of suitable valves are e.g. solenoid valves or pinch valves. Pinch valves are preferred because they can be equipped with single use hoses, so that the parts of the pinch valves coming in contact with the liquid medium or the watery formulation can be exchanged easily. Hereby the device can be made ready for a new and different liquid medium and pollution and/or contamination can be prevented.

FIG. 2 shows a schematic view on a device for producing liquid media for cell cultures according to the present invention. The device comprises a mixing vessel 10 in which a watery formulation (not shown in FIG. 2) can be mixed to produce a liquid medium for cell culture growth. The top of the mixing vessel 10 can be closed by a lid 12, which may be openable. The lid 12 may seal the mixing vessel 10, preferably in a gas tight manor and/or in a pressure tight manor. The bottom of the mixing vessel 10 can be connected via an outlet to a pipe 14. The outlet to the pipe 14 is arranged on the lowest part of the mixing vessel 10 to allow all fluid from the mixing vessel 10 to be drained or pumped from the mixing vessel 10. The connection to the pipe 14 can preferably be opened and closed by means of an outlet valve 16, which may be controlled automatically or manually.

An agitator 18 can be arranged inside the mixing vessel 10 to mix the watery formulation (not shown) therein. The agitator 18 can be driven by a motor 20 via an axis 22. The agitator 18 can comprise a multitude of mixing blades. The mixing blades can be arranged on and fastened to the axis 22. Alternatively, the agitator 18 could also comprise permanent magnets and thus be driven by changing magnetic fields penetrating the mixing vessel 10 or created within the mixing vessel 10.

A pH meter 24 and a dissolutions sensor 26 can be arranged inside the mixing vessel 10 to measure the condition of the watery formulation therein.

Furthermore, an osmolarity sensor 28 for measuring the osmolarity of the liquid inside the mixing vessel 10 can be arranged therein. The dissolution sensor 26 can preferably be a turbidity sensor. Further sensors useful for characterizing the watery formulation in the mixing vessel 10, such as temperature sensors, conductivity sensors, viscosity sensors, turbidity sensors, chromatographs, pressure sensors, liquid level sensors and the like may be provided additionally.

A dosing apparatus 30 for dosing a specific amount of a powdered or granulate ingredient or mixture of ingredients can be connected to the mixing vessel 10. The dosing apparatus 30 can comprise at least one container 32 for storing the ingredients. The dosing apparatus 30 is designed to dose specific amounts of the ingredients or mixtures of ingredients into the mixing vessel 10 to be mixed with the watery formulation or the water therein. The dosing apparatus 30 can be connected to the mixing vessel 10 via an outlet 36. The dosing apparatus comprises a motor 34 for generating a movement within the dosing apparatus required for moving powdered or granulate ingredient or mixture of ingredients.

A water supply 40 is connected to the mixing vessel 10. The water supply can be designed to fill a specific amount of water into the mixing vessel 10. Furthermore, an acid supply 42, a buffer supply 44 and a base supply 46 can be connected to the mixing vessel 10 to add specific amount of acid, watery acid, buffer, buffer solution, base and/or watery base to the watery formulation inside the mixing vessel 10. The pipe 14 is connected to a flow generating apparatus 48 for controlling a flow of watery formulation from the mixing vessel 10. The flow generating apparatus 48 can comprise a pump for generating the volume flow from the mixing vessel 10 to a bioreactor (not shown in FIG. 2 but can be similar to the one shown in FIG. 1).

A control system 50 can be provided to control the speed of motor 20, the kind, mixture and amount of ingredients provided by the dosing apparatus into the mixing vessel 10, the amount of water applied by the water supply 40 into the mixing vessel 10, the amount of acid or watery acid applied by the acid supply 42 into the mixing vessel 10, the amount of buffer solution applied by the buffer supply 44 into the mixing vessel 10 and the amount of base or watery base applied by the base supply 46 into the mixing vessel 10. For this purpose, the control system 50 can be connected to the motor 20, the dosing apparatus 30, the water supply 40, the acid supply 42, the buffer supply 44 and the base supply 46 and can be programmed to control the functions thereof. In addition, the control system 50 can be connected to the pH meter 24, the dissolution sensor 26 and the osmolarity sensor 28 to read measured values of the pH, measured values from the dissolution sensor 26 representing the dissolution (for example by reading a value of the turbidity of the watery formulation by means of a turbidity sensor as the dissolution sensor) and of the osmolarity of the watery formulation. The control system 50 can have access to or can comprise a timing element (not shown) to control the functions of the device depending on time information.

The control system 50 can be programmed to control the functions of the device depending from the measured values of all sensors it has access to. Thereby, it is possible to control the mixing process of the watery formulation inside the mixing vessel 10 and the supply of watery formulation or ready mixed liquid (cell culture) media via the flow generating apparatus 48.

The watery formulation can be pumped by the flow generating apparatus 48 through two valves 52, 54 and a sterile filter 56 installed between the two valves 52, 54. The two valves 52, 54 can be used to easily allow exchanging the sterile filter 56. An outlet 58 with a connecting pipe 60 can be installed in the line behind the sterile filter 56. The outlet 58 can be used as tapping point to draw samples of the filtered liquid media.

All parts of the device can be held by a holding frame 62, to which all the parts are fastened. A casing (not shown) can be used to protect the parts.

A flush valve 64 can be arranged in the lines behind the sterile filter 56 to allow the mixing vessel 10, the pipe 16 and the lines connecting the flush valve 64 to the mixing vessel 10 and to the water supply 40 with water and/or buffer solution from the buffer supply 44, to clean these parts from residues of earlier mixing processes. The filtered liquid medium can be pumped by the flow generating apparatus 48 through an outlet pipe 66, by which the liquid medium can be delivered to a bioreactor (not shown in FIG. 2).

In the following an embodiment for a process for producing liquid media for cell culture growth according to the present invention is described. The process is described using the device according to FIG. 2. The whole process can be and preferably is controlled by the control system 50, which is thus programmed to control the process as described.

First water can be filled by the water supply 40 into the mixing vessel 10. Only a part of the desired final volume is filled into the mixing vessel 10 to precisely control the desired amount of watery formulation and liquid medium at a later stage. Typically, this is between 50 and 90%, more preferred around 70% to 85% of the desired final volume. Next the agitation can be started by revolving the agitator 18 in the mixing vessel 10. While stirring the water in the mixing vessel 10, dry powder or dry granulate of one or more ingredients can be poured into the mixing vessel 10 by means of the dosing apparatus 30. The ingredient(s) at least partly dissolve(s) in the stirred water to form a watery formulation.

While dissolving the ingredients in the watery formulation the pH can be measured by the pH meter 24 and the progress of dissolution can be measured by the dissolution sensor 26 (for example by measuring the turbidity). The osmolarity can be measured by the osmolarity sensor 28. In addition, also the temperature, the electrical conductivity and other physical properties like viscosity, pressure, liquid level and the like can also be measured. All measured values can be evaluated by the control system 50 to adapt the pH, control the stirring (speed and/or time) and the addition of further ingredients (point of time, mixture of ingredients and amount) to the watery formulation.

For example, after stirring for a while after introducing the first batch of ingredients, the pH can be lowered for example to 4.5 by filling acid or watery acid into the mixing vessel 10 by means of the acid supply 42. The lower pH of the watery formulation allows or helps to dissolve another second part of the ingredients in the watery formulation. The amount of acid or watery acid is controlled by data from the pH meter to precisely set a certain pH in the watery solution. As soon as the signals from the dissolution sensor 26 and/or the osmolarity sensor signal the control system 50 that the second part of the ingredients have been dissolved in the watery formulation, a base or a watery base or a bicarb solution can be added to the watery formulation by means of the base supply 46 or the buffer supply 44. This will allow to dissolve ingredients which require a higher pH to dissolve. Based on these signals from the sensors it is also possible to add further ingredients by means of the dosing apparatus 30 to the watery formulation in the mixing vessel 10 before or even while adding an acid, a watery acid, a base, a watery base or a buffer solution.

In a next step again acid or watery acid can be filled into the mixing vessel 10 depending from a duration of time stirring and/or depending from data from the dissolution sensor 26 or from the osmolarity sensor 28. A buffer solution can be added to the watery formulation by means of the buffer supply 44 to adjust the pH of the watery formulation in a next step.

FIG. 3 shows a measured example pH of a watery formulation during a process for producing liquid media for cell culture according to the present invention, FIG. 4 shows a measured example pH and electrical conductivity of a watery formulation during a process for producing liquid media for cell culture according to the present invention and FIG. 5 shows an example for measured salinity and electrical conductivity of a watery formulation during an exemplary process for producing liquid media for cell culture according to the present invention.

When the watery formulation in the mixing vessel 10 is satisfactory, for example because all ingredients are dissolved (measured by the dissolution sensor 26), the pH has the required value (measured by the pH meter 24), the electrical conductivity has the required value (measured by a sensor for measuring the electrical conductivity of the watery formulation) and/or the osmolarity has the desired value (measured by the osmolarity sensor 28), the final volume of the watery formulation is filled up by means of the water supply 40 to the desired amount or volume. Then again, the watery formulation can be controlled by means of the sensors 24, 26, 28 to control the quality of the watery formulation or watery solution.

In a next step a sterile filtration can take place by pumping the watery formulation or watery solution by means of the flow generating apparatus 48 through the sterile filter 56. The flow generating apparatus 48 can comprise an electric pump but the watery formulation may also be driven by gravity and the flow generating apparatus 48 may comprise a controllable valve therefore alternatively or additionally to an electric pump. The filtered watery formulation or watery solution can be used as cell culture media in a bioreactor like the one shown in FIG. 1. For documentation a part of the liquid medium can be saved by tapping a part of the watery formulation or watery solution from the outlet 58.

After all the watery solution or watery formulation (apart from remaining residuals) has been pumped from the mixing vessel 10, the device can be cleaned and the bioburden can be reduced by rinsing the mixing vessel 10 and all pipes 14, valves 16, 52, 54 and the flow generating apparatus 48. To avoid contamination, especially if a new type of liquid medium shall be produced using the same device, all single use parts can be exchanged. Preferably the pipes 14 and valves 16, 52, 54 coming into contact with the watery formulation can be or can contain single use parts.

The features of the invention disclosed in the above description, the claims, figures, and exemplary embodiments can be essential both individually and in any combination for implementing the various embodiments of the invention.

### LIST OF REFERENCE SYMBOLS

1 Bioreactor
2 Cell culture
3 Stirrer
4 Cell retention device
10 Mixing vessel
12 Lid
14 Pipe
16 Outlet valve
18 Agitator/Mixing blade
20 Motor
22 Axis
24 pH meter
26 Dissolution sensor
28 Osmolarity sensor
30 Dosing apparatus
32 Container
34 Motor
40 Water supply/Water inlet
42 Acid supply
44 Buffer supply
46 Base supply
48 Flow generating apparatus
50 control system
52 Valve
54 Valve
56 Sterile filter
58 Outlet
60 Connecting pipe
62 Holding frame
64 Flush valve
66 Outlet pipe

The entire disclosure of all applications, patents, and publications cited above and below as well as European patent application EP20184241.6, filed on Jul. 6, 2020, are hereby incorporated by reference.

1. A system for performing cell culture comprising a bioreactor, a holding tank, a device for producing liquid media for cell cultures and a control system connected to the bioreactor, the holding tank and the device for producing liquid media for cell cultures, whereby the liquid media are produced by dissolving ingredients in water, the device for producing liquid media for cell cultures comprising a mixing vessel for holding and mixing a watery formulation;
an agitator for mixing the watery formulation in the mixing vessel;
at least one pH meter in the mixing vessel or in fluid connection with the mixing vessel;
a dosing apparatus connected to the mixing vessel for filling a specific amount of at least one solid ingredient or of at least one mixture of solid ingredients into the mixing vessel;
a water supply for adding water into the mixing vessel;
a base supply for adding a specific amount of a base or a watery base to the mixing vessel;
an acid supply for adding a specific amount of an acid or a watery acid to the mixing vessel;
a flow generating apparatus for generating or allowing a flow of the watery formulation from the mixing vessel;
the control system being connected to the pH meter, such that the measured values of the pH meter are accessible by the control system, the control system being connected to the water supply to control the amount of water being filled into the mixing vessel, the control system being connected to the base supply to control the amount of base or watery base being filled into the mixing vessel, the control system being connected to the acid supply to control the amount of acid or watery acid being filled into the mixing vessel, the control system being connected to the dosing apparatus to control the amount of the at least one ingredient or the at least one mixture of ingredients being filled into the mixing vessel, whereby the control system is programmed to control the dosing apparatus, the water supply, the base supply, the acid supply, and preferably the flow generating apparatus, depending from the measured values of at least one of the pH meter
and whereby the bioreactor and the mixing vessel of the device for producing liquid media for cell cultures are connected via at least a pipeline or tube which is interrupted by at least one holding tank.

2. A system for performing cell culture according to claim 1 characterized in that at least one sterile filter is arranged in said pipeline or tube.

3. A system for performing cell culture according to claim 1 characterized in that the system comprises a signal receiving unit which is able to receive a signal from the bioreactor, and the control system is programmed to control the flow of the watery formulation in response to the signal received from the bioreactor via the signal receiving unit.

4. A system for performing cell culture according to claim 1, characterized in that the bioreactor comprises a controller and a signal sending unit which sends a signal to the signal receiving unit if the amount of watery formulation drops below a certain value or fresh watery formulation is needed.

5. A system for performing cell culture according to claim 1, characterized in that the holding tank comprises a controller and a signal sending unit which sends a signal to the signal receiving unit if the amount of watery formulation drops below a certain value or fresh watery formulation is needed and the control system is programmed to control the production of the watery formulation in response to the signal received from the holding tank via the signal receiving unit.

6. A system for performing cell culture according to claim 1, characterized in that the bioreactor is operated in perfusion mode.

7. A system according to claim 1 comprising at least one dissolution sensor for detecting the presence of undissolved ingredients in the watery formulation, whereby the control system is connected to the at least one dissolution sensor, such that the measured values of the at least one dissolution sensor are accessible by the control system.

8. A system according to claim 1 the device comprising at least one sensor for measuring the electrical conductivity of the watery formulation, whereby the at least one sensor for measuring the electrical conductivity of the watery formulation is located in the mixing vessel and/or in a pipe for conducting the flow from the mixing vessel, whereby the control system is connected to the at least one sensor for measuring the electrical conductivity of the watery formulation, such that the measured values of the at least one sensor for measuring the electrical conductivity of the watery formulation are accessible by the control system and the control system being designed to control at least the flow generating apparatus depending from the measured values of the at least one sensor for measuring the electrical conductivity of the watery formulation, and/or
a sensor for measuring the osmolarity of the watery formulation, whereby the sensor for measuring the osmolarity of the watery formulation is located in the mixing vessel and/or in a pipe for conducting the flow from the mixing vessel, whereby the control system being connected to the sensor for measuring the osmolarity of the watery formulation, such that the measured values of the sensor for measuring the osmolarity of the watery formulation are accessible by the control system and the control system being designed to control at least the flow generating apparatus depending from the measured values of the sensor for measuring the osmolarity of the watery formulation and/or
a sensor for level indication of the watery formulation inside the mixing vessel, whereby the control system being connected to the sensor for level indication, such that the measured values of the sensor for level indication are accessible by the control system and the control system being designed to control at least the flow generating apparatus depending from the measured values of the sensor for level indication and/or
a volume sensor and/or a liquid level sensor to measure the volume of watery formulation in the mixing vessel, whereby the control system being connected to the volume sensor and/or the liquid level sensor to control the amount of watery formulation in the mixing vessel and being programmed to fill water and/or the at least one ingredient or the at least one mixture of ingredients into the mixing vessel depending from the measured value of the volume sensor and/or the liquid level sensor and/or
at least one weight sensor to measure the weight of content in the mixing vessel and/or the amount of the at least one ingredient or of the at least one mixture of ingredients to be filled into the mixing vessel by the dosing apparatus, whereby
the control system being connected to the at least one weight sensor to control the amount of watery formulation in the mixing vessel and being programmed to fill water and/or the at least one ingredient or the at least one mixture of ingredients into the mixing vessel depending from the measured value of the at least one weight sensor and/or the control system being connected to the at least one weight sensor to control the weight of the at least one ingredient or of the at least one mixture of ingredients to be filled into the mixing vessel by the dosing apparatus and being programmed to fill water and/or additional of the at least one ingredient or the at least one mixture of ingredients into the mixing vessel depending from the weight measured by the at least one weight sensor.

9. A system according to claim 1, whereby the flow generating apparatus is or comprises a pumping device for pumping the formulation from the mixing vessel to the holding tank and/or from the holding tank into the bioreactor and thereby generating the flow of the formulation from the mixing vessel and/or the flow generating apparatus is or comprises a controllable valve for controlling the flow of the formulation from the mixing vessel to the holding tank and/or from the holding tank into the bioreactor, whereby preferably the flow of the formulation is driven by gravity and/or the pumping device.

10. A system according to claim 1, whereby the control system comprises one or more of the following programming:
the control system is programmed to control the agitator depending from the measured values of at least one of the pH meter and/or, if present, of the dissolution sensor, and/or of the at least one sensor for measuring the electrical conductivity of the watery formulation and/or of the sensor for measuring the osmolarity of the watery formulation and/or a time information given by the timing element;
the control system is programmed to produce at least two different types of liquid media for the growth of at least two different cell cultures or different process phases of the same cell culture process;
the control system is programmed to start the production of a new batch of liquid media triggered via a signal or a request to the control system via at least one interface.

11. A system according to claim 1, whereby all pipes and containers coming in contact with the watery formulation of the system are single-use parts.

12. A system according to claim 1, whereby the flow of the watery formulation from the mixing vessel generated or allowed by the flow generating apparatus flows into the holding tank and/or into the bioreactor, whereby the control system is connected to a signal receiving unit, which is able to receive a signal from the bioreactor and/or the holding tank, and the control system is programmed to control the flow of the watery formulation in response to the signal received from the bioreactor and/or the holding tank via the signal receiving unit.

13. A process for performing cell culture comprising the following method steps:
Providing a system for performing cell culture comprising a bioreactor, a holding tank and a device for producing liquid media for cell cultures according to claim 1
Performing cell culture in a bioreactor
Continuously or one or several times during the cell culture performing a process for producing liquid media for cell culture using a device for producing liquid media for cell culture
Continuously or one or several times during the cell culture flowing watery formulation, which typically is liquid medium for cell culture produced in the device for producing liquid media for cell culture from the mixing vessel of the device to the holding tank and/or from the holding tank to the bioreactor,
whereby the process for producing liquid media for cell culture comprises the steps of
A) Filling water and a specific amount of at least one ingredient or at least one mixture of ingredients into the mixing vessel and mixing them therein to a watery formulation;
B) Once or repeatedly measuring pH of the watery formulation in the mixing vessel by the at least one pH meter and optionally detecting the presence of undissolved ingredients in the watery formulation in the mixing vessel at least one dissolution sensor by means of the control system;
C) Automatically filling at least once a specific amount of water, base, watery base, acid, watery acid and/or one or more buffer solutions into the mixing vessel depending on the measured pH value and/or the presence of undissolved ingredients by means of the control system;
D) After all of the required at least one ingredient or the at least one mixture of ingredients have been filled into the mixing vessel and mixed to a final watery formulation, providing a volume flow of the final watery formulation from the mixing vessel controlled by the control system.

14. A process according to claim 13, whereby the process for producing media further comprises the method step
C2) Automatically filling a specific amount of one or more selected from an additional ingredient, an additional mixture of ingredients, water, a base, a watery base, one or more buffer solutions, an acid and a watery acid, into the mixing vessel controlled by the control system before method step D).

15. A process for cell culture, comprising a process according to claim 13, further comprising culturing cells in a bioreactor with liquid media inlet and a harvest outlet, comprising the method steps of
i. continuously or one or several times during the cell culture process inserting fresh liquid media from the device into the bioreactor via the liquid media inlet; and
ii. continuously or one or several times during the cell culture process removing harvest from the bioreactor via the harvest outlet;
and wherein preferably the process steps i and ii are regulated such that the volume of the cell culture in the bioreactor is kept at a constant level, and
a sensor for measuring the liquid level in the bioreactor automatically sends a signal to the control system if the level of liquid media drops below a predefined value or if harvest is removed from the bioreactor, whereby the control system starts preparation of fresh liquid medium according to the process upon receiving the signal.
